(19) United States
(12) Patent Application Publication
(10) Pub. No.: US 2007/0043126 A1
(43) Pub. Date: Feb. 22, 2007

(54) Methanol synthesis and reaction system

(76) Inventor: James R. Lattner, LaPorte, TX (US)

Correspondence Address: ExxonMobil Chemical Company;Law Technology, P.O. Box 2149, Baytown, TX, 77522-2149, US

(21) Appl. No.: 11/484,304

(22) Filed: Jul. 11, 2006

Related U.S. Application Data
(60) Provisional application No. 60/709,223, filed on Aug. 18, 2005.

(51) Int. Cl. C07C 27/26

(52) U.S. Cl. 518/726

(57) ABSTRACT

This invention is directed to a process for making a methanol product from a synthesis gas (syngas) feed. Preferably, the process uses a reaction system with three reactors. Particular benefits of a three reactor system are provided by arranging each of the reactors in series, with at least one of the reactors being cooled by evaporative cooling.

### CROSS REFERENCE TO RELATED APPLICATIONS

This application claims the benefit of Provisional Application No. 60/709,223 filed Aug. 18, 2005, the disclosure of which is fully incorporated herein by reference.

### FIELD OF THE INVENTION

This invention relates to the production of methanol. In particular, this invention relates to the production of methanol using a three reactor system.

### BACKGROUND OF THE INVENTION

Current technology limits the size of a single train methanol plant to about 5000 t/day. There is incentive to increase the scale of methanol production to enable economic conversion of remote natural gas into a transportable form, either for fuel needs or for other processes such as methanol-to-olefins (MTO) processes. Such prospects may entail increasing methanol production on a scale of from 2 to 4 times current processes.

Many methanol synthesis reactor designs currently utilize multiple reactors with multiple cooling steps. See Appl, M., Modern Production Technologies, British Sulphur Publishing, London, 1997 ISBN 1 8733387 26 1. Generally, the cooling takes place in at least two ways: (i) several adiabatic reactor beds in series with coolers in between stages, or (ii) cooling tubes located within a fixed reactor bed.

WO 2004/065341 discloses a process for making methanol in which the methanol is synthesized from pre-heated methanol synthesis gas in one or more adiabatic synthesis stages with cooling of the resultant gas after each stage. Further methanol synthesis is then effected on the resultant partially reacted synthesis gas in a bed of synthesis catalyst cooled by means of a coolant flowing co-currently through tubes disposed in the catalyst bed.

What is needed is a reaction system that is capable of operating at a substantially high temperature and recovering heat from the reaction system in the most efficient manner. The reaction system should also be operated within a temperature profile that closely follows a maximum rate of conversion of reactants to provide an efficient overall reaction process.

### SUMMARY OF THE INVENTION

This invention provides a process for making methanol from synthesis gas (syngas). The process is carried out at a temperature profile that closely follows a maximum rate of conversion of reactants to provide an efficient overall reaction process. In addition, the process provides for an efficient recovery of heat that is generated from the exothermic conversion of reactants to methanol product.

According to one aspect of the invention, there is provided a process for making methanol from syngas in a three reactor system. The process includes feeding syngas to three reactors in series to form the methanol product. The reactors are cooled during the formation of the methanol product such that at least one of the reactors is cooled by evaporative cooling.

In one embodiment of the invention, at least one of the reactors is cooled by water evaporative cooling. In another embodiment, at least one reactor is cooled by preheating the syngas feed. In yet another, at least one reactor is cooled by water sensible cooling. More preferably, one reactor is cooled by water evaporative cooling, one by preheating syngas feed, and one by water sensible cooling.

In another embodiment, the first of the three reactors in series is cooled by evaporative cooling. Preferably, the first of the three reactors in series is cooled by water evaporative cooling.

In yet another embodiment, the third reactor in series is cooled by water sensible cooling. Preferably, the second and third reactors in series are cooled by sensible cooling. More preferably, the second reactor in series is cooled by gas sensible cooling. In one particular embodiment, the second reactor in series is cooled by preheating the syngas feed to the first reactor.

The first reactor preferably has an inlet temperature of from 230° C. to 270° C. Preferably, the first reactor also has an outlet temperature of from 245° C. to 285° C.

In one embodiment, the outlet temperature of the second reactor is cooler than that of the first reactor. Preferably, the outlet temperature of the second reactor is at least 10° C. cooler than that of the first reactor.

In another embodiment, the third reactor has an outlet temperature of from 170° C. to 210° C. Preferably, the outlet temperature of the third reactor is cooler than that of the second reactor. More preferably, the outlet temperature of the third reactor is at least 20° C. cooler than that of the second reactor.

In one embodiment of the invention, at least one reactor is a fixed bed reactor having multiple cooling tubes spaced within a catalyst bed. Preferably, at least one reactor contains multiple tubes, each packed with catalyst and surrounded by a heat transfer medium.

The process is carried out at a pressure that provides efficient recovery of heat. Preferably, the process is carried out at a pressure of less than 110 bara.

### BRIEF DESCRIPTION OF THE DRAWINGS

FIG. 1 is a flow diagram of one embodiment of a reaction system that can be used to carry out the process of the invention.

FIG. 2 shows temperature profiles for the three reactor system depicted in FIG. 1 according to the conditions carried out in Example 1.

FIG. 3 shows conversion profiles for the three reactor system of Example.

FIG. 4 shows the temperature profile for the single reactor system of Example 2.

FIG. 5 shows the temperature profile for the single reactor system of Example 3.

### DETAILED DESCRIPTION OF THE INVENTION

### I. ACHIEVING MAXIMUM RATE OF CONVERSION

This invention is directed to a process for making a methanol product from a synthesis gas (syngas) feed. The process uses a reaction system that incorporates three reactors in series.

The process of making the methanol from syngas is an exothermic reaction, which means that the reaction gives off heat. The invention provides for conversion of the reactants at very close to the maximum rate of conversion, while also recovering the reaction heat in an efficient and useful manner. One benefit of such a conversion and recovery process is that less catalyst and smaller reactor volumes can be used to achieve a desired conversion of syngas to methanol relative to known systems. Alternatively, an advantage is that greater CO and CO₂ conversion can be obtained using comparable amounts of catalyst relative to known systems.

The reactors used in this invention can be of any type capable of converting syngas reactants to methanol product. Preferably, the reactors are fixed bed reactors with provisions for heat transfer within the catalyst bed(s). The catalyst may also be fluidized, with either gas or with a gas/liquid mixture. In one embodiment, at least one reactor is a fixed bed reactor having multiple cooling tubes (e.g., coils) spaced within a catalyst bed. In another embodiment, at least one reactor contains multiple tubes, each packed with catalyst, and surrounded by a heat transfer medium.

The benefits of the invention are provided by arranging each of the reactors in series, with at least one of the reactors being cooled by evaporative cooling. Evaporative cooling means that the particular reactor is cooled using a fluid that changes from the liquid state to the vapor state during the cooling process. The liquid that is used for the evaporative cooling is preferably water, although any other liquid having appropriate heat transfer, boiling point, and safety qualities can be used.

Sensible cooling is also used in this invention. Sensible cooling can also be referred to as sensible heat transfer cooling. Sensible cooling is accomplished using a fluid to transfer heat such that the fluid does not change its physical phase. For example, a gas can be used as a heat transfer medium, without the gas being changed to a liquid or solid in the vessel in which the heat transfer occurs. In addition, a liquid can be used as a heat transfer fluid, without the liquid being changed to a vapor or solid in the vessel in which heat transfer occurs.

The invention is preferably carried out in a three reactor system. More preferably, the three reactors are connected in series.

In this invention, at least one of the reactors is cooled by evaporative cooling. Preferably, the remaining reactors are cooled by sensible cooling.

The predominant means of cooling in this invention is by supplying fluid to the reactor itself. Such a reactor can include appropriate internal or external tubes to enable the transfer of heat from the reaction medium to the cooling fluid during the formation of the methanol product. Although heat transfer devices such as heat exchangers can be used between reactors, such devices are not necessary. Thus, the invention includes embodiments of having reactors in series without having heat exchangers between the reactors.

The reactors that are used in making the methanol product can be any type of reactor capable of holding methanol forming catalyst and converting syngas reactants to methanol product. Preferred are reactors that have a provision for transfer of heat within a bed of catalyst. Particularly preferred reactors are fixed bed reactors with heat transfer tubes or coils within the catalyst beds. The catalyst may be either inside or outside of the heat transfer tubes, with the cooling fluid on the other side. Multiple cooling tubes can be included within a single reactor.

In a preferred embodiment of the invention, the reaction system incorporates three reactors in series. Preferably, the first of the three reactors in series is cooled by evaporative cooling. More preferably, the first of the three reactors in series is cooled by water evaporative cooling. The designation of first, second and third reactors is based on the order in which catalytic conversion occurs. For example, reactor 1 would be the first reactor in which syngas feed contacts methanol forming catalyst to begin the reaction process.

In one embodiment of this invention, the effluent from any one or more reactors can be used to preheat the syngas feed. In a particular embodiment, the effluent from one reactor can be flowed to a reactor having a shell and tube arrangement to preheat the syngas feed. In such an embodiment, the effluent of one reactor can be flowed through the shell side of a reactor where the effluent encounters methanol forming catalyst and the syngas can be flowed through the tube side such that the syngas is preheated as a result of the exothermic reaction. Likewise, the effluent of one reactor can be flowed through the tube side of a reactor where the effluent encounters methanol forming catalyst, and the syngas can be flowed through the shell side such that the syngas is preheated. In one embodiment, the effluent of the first reactor is used to preheat syngas that is flowed through the second reactor. In other words, the second reactor is cooled by preheating the syngas feed in the second reactor. Preferably, the syngas flows through the tube side of the second reactor and the catalyst is on the shell side.

It is preferred in one embodiment, that at least one of the reactors in the reaction system is cooled by water sensible cooling. Preferably, the third reactor in series is cooled by water sensible cooling.

In one embodiment of the invention, two of the reactors in the system are cooled by sensible cooling. Preferably, the second and third reactors in a reactor series are cooled by sensible cooling. In one embodiment, the second reactor in series is cooled by gas sensible cooling. In another embodiment, the second reactor in series is cooled by effluent product from the first reactor in series.

It is preferred in this invention to keep the reaction temperatures in a range that allows for the maximum rate of conversion under continuous steady state conditions. This typically means that each of the reactors will be run continuously at different temperatures. Relative reactor temperature can be evaluated using average reactor temperature or reactor outlet temperature. Satisfying both conditions is not required.

In one embodiment, the outlet temperature of the third reactor in series is cooler than the outlet temperature of the second reactor. In another embodiment, the outlet temperature of the second reactor is cooler than the outlet temperature of the first reactor.

It is preferred that the third reactor have an outlet temperature that is at least 20° C. cooler than the outlet temperature of the second reactor. Preferably, the third reactor has an outlet temperature that is at least 30° C., more preferably at least 40° C., cooler than that of the second reactor.

In another embodiment, the second reactor has an outlet temperature that is at least 10° C. cooler than the outlet temperature of the first reactor. Preferably, the second reactor has an outlet temperature that is at least 20° C., more preferably at least 25° C., cooler than that of the first reactor.

Each of the reactors in the three reactor series is preferably operated in a temperature range that allows for maximum rate conversion of reactants, e.g., CO and CO₂. It is of particular interest to maintain reactor temperature in each reactor of the reaction system so as to use as little catalyst as possible for the volume of product desired.

Reactor temperature is preferably controlled by monitoring reactor outlet temperature. Control over temperature outlet can be achieved in several ways including controlling reactor inlet temperature, the amount of catalyst in the reactor and the cooling characteristics of the cooling medium, including rate of flow. In a preferred embodiment of this invention, heat exchangers between reactors are not used. In preferred series arrangement, the effluent from reactor 1 serves as the feed to reactor 2, and the effluent from reactor 2 serves as the feed to reactor 3, with no cooling applied between reactors.

In one embodiment, syngas is sent to the first reactor at a reactor inlet temperature of from 230° C. to 270° C. More preferably, is syngas sent to the first reactor at an inlet temperature of from 235° C. to 265° C., most preferably from 245° C. to 255° C.

In another embodiment of the invention, the first reactor is maintained at an outlet temperature of from 245° C. to 285° C. Preferably, the first reactor is maintained at an outlet temperature of from 250° C. to 280° C., more preferably from 260° C. to 270° C.

In yet another embodiment of the invention, the second reactor is maintained at an outlet temperature of from 255° C. to 265° C. Preferably, the second reactor is maintained at an outlet temperature of from 250° C. to 255° C., more preferably from 240° C. to 245° C.

In another embodiment, the third reactor is maintained at an outlet temperature of from 170° C. to 210° C. More preferably, the third reactor is maintained at an outlet temperature of from 175° C. to 205° C., more preferably from 185° C. to 195° C.

The reaction system can be operated at any pressure in which product is capable of being produced. Preferred pressures are relatively low methanol synthesis pressures. The pressures should be sufficiently low to reduce equilibrium conversion limitations at the outlet of the reactor, where conversion is not as efficient compared to the reactor inlet. The result of a low pressure operation is that product is produced at relatively low final reactor outlet temperatures. A low final reactor outlet temperature-operation in a three reactor system provides a particularly preferred advantage in the low pressure scheme. Preferably, the reaction process is carried out at a pressure of less than 110 bara. More preferably, the process is carried out at a pressure of less than 90 bara, and most preferably less than 70 bara.

### II. SYNGAS FEED DESCRIPTION

Synthesis gas (syngas) is used in the feed to the reaction system of this invention. Desirably, the synthesis gas feed (including any recycle syngas recovered from the process itself as well as fresh syngas) has a molar ratio of hydrogen (H₂) to carbon oxides (CO+CO₂) in the range of from about 0.5:1 to about 20:1, preferably in the range of from about 1:1 to about 10:1. In another embodiment, the synthesis gas has a molar ratio of hydrogen (H₂) to carbon monoxide (CO) of at least 2:1. Carbon dioxide is optionally present in an amount of not greater than 50% by weight, based on total weight of the synthesis gas, and preferably less than 20% by weight, more preferably less than 10% by weight.

Desirably, the stoichiometric molar ratio is sufficiently high so as maintain a high yield of methanol, but not so high as to reduce the volume productivity of methanol. Preferably, the synthesis gas fed to the methanol synthesis process has a stoichiometric molar ratio (i.e., a molar ratio of (H₂—CO₂)/(CO+CO₂)) of from about 1.0:1 to about 2.7:1, more preferably from about 1.5 to about 2.5, more preferably a stoichiometric molar ratio of from about 1.7:1 to about 2.5:1.

### III. CATALYST DESCRIPTION

Preferably, the methanol synthesis catalyst used in the process of this invention includes an oxide of at least one element selected from the group consisting of copper, silver, zinc, boron, magnesium, aluminum, vanadium, chromium, manganese, gallium, palladium, osmium and zirconium. More preferably, the catalyst is a copper based catalyst, more preferably in the form of copper oxide.

In another embodiment, the catalyst used in the methanol synthesis process is a copper based catalyst, which includes an oxide of at least one element selected from the group consisting of silver, zinc, boron, magnesium, aluminum, vanadium, chromium, manganese, gallium, palladium, osmium and zirconium. Preferably, the catalyst contains copper oxide and an oxide of at least one element selected from the group consisting of zinc, magnesium, aluminum, chromium, and zirconium. More preferably, the catalyst contains oxides of copper and zinc.

In yet another embodiment, the methanol synthesis catalyst comprises copper oxide, zinc oxide, and at least one other oxide. Preferably, the at least one other oxide is selected from the group consisting of zirconium oxide, chromium oxide, vanadium oxide, magnesium oxide, aluminum oxide, titanium oxide, hafnium oxide, molybdenum oxide, tungsten oxide, and manganese oxide.

In various embodiments, the methanol synthesis catalyst comprises from about 10 wt % to about 70 wt % copper oxide, based on total weight of the catalyst. Preferably, the methanol synthesis contains from about 15 wt % to about 68 wt % copper oxide, and more preferably from about 20 wt % to about 65 wt % copper oxide, based on total weight of the catalyst.

In one embodiment, the methanol synthesis catalyst comprises from about 3 wt % to about 30 wt % zinc oxide, based on total weight of the catalyst. Preferably, the methanol synthesis catalyst comprises from about 4 wt % to about 27 wt % zinc oxide, more preferably from about 5 wt % to about 24 wt % zinc oxide.

In embodiments in which copper oxide and zinc oxide are both present in the methanol synthesis catalyst, the ratio of copper oxide to zinc oxide can vary over a wide range. Preferably in such embodiments, the methanol synthesis catalyst comprises copper oxide and zinc oxide in a Cu:Zn atomic ratio of from about 0.5:1 to about 20:1, preferably from about 0.7:1 to about 15:1, more preferably from about 0.8:1 to about 5:1.

### IV. RECOVERY AND FURTHER PROCESSING OF METHANOL PRODUCT

The methanol product from the final, preferably third, reactor is generally sent to a separation unit or vessel to remove light product having a higher boiling point than the methanol. This separation preferably yields a liquid product rich in methanol, although the separated methanol product can include other components such as water. The separated methanol product can be used “as is,” or it can be further processed if desired. Processing can be accomplished using any conventional means. Examples of such means include distillation, selective condensation, and selective adsorption. Process conditions, e.g., temperatures and pressures, can vary according to the particular methanol composition desired. It is particularly desirable to minimize the amount of water and light boiling point components in the methanol composition, but without substantially reducing the amount of methanol present.

In one embodiment, the separated and recovered methanol product is sent to a let down vessel so as to reduce the pressure to about atmospheric or slightly higher. This let down in pressure allows undesirable light boiling point components to be removed from the methanol composition as a vapor. The vapor is desirably of sufficient quality to use a fuel.

In another embodiment, the separated recovered methanol product is sent from the methanol synthesizing unit or vessel to a distillation system. The distillation system contains one or more distillation columns which are used to further separate the desired methanol composition from water and hydrocarbon by-product streams. Desirably, the methanol composition that is separated from the crude methanol comprises a majority of the methanol contained in the methanol product prior to separation.

In one embodiment, the distillation system includes a step of treating the recovered methanol product steam being distilled so as to remove or neutralize acids in the stream. Preferably, a base is added in the system that is effective in neutralizing organic acids that are found in the methanol stream. Conventional base compounds can be used. Examples of base compounds include alkali metal hydroxide or carbonate compounds, and amine or ammonium hydroxide compounds. In one particular embodiment, about 20 ppm to about 120 ppm w/w of a base composition, calculated as stoichiometrically equivalent NaOH, is added, preferably about 25 ppm to about 100 ppm w/w of a base composition, calculated as stoichiometrically equivalent NaOH, is added.

Examples of distillation systems include the use of single and two column distillation columns. Preferably, the single columns operate to remove volatiles in the overhead, methanol product at a high level, fusel oil as vapor above the feed and/or as liquid below the feed, and water as a bottoms stream.

In one embodiment of a two column system, the first column is a “topping column” from which volatiles are taken overhead and methanol liquid as bottoms. The second is a “rectifying column” from which methanol product is taken as an overhead stream or at a high level, and water is removed as a bottoms stream. In this embodiment, the rectifying column includes at least one off-take for fusel oil as vapor above the feed and/or as liquid below the feed.

In another embodiment of a two column system, the first column is a water-extractive column in which there is a water feed introduced at a level above the crude methanol feed level. It is desirable to feed sufficient water to produce a bottoms liquid containing over 40% w/w water, preferably 40% to 60% w/w water, and more preferably 80% to 95% w/w water. This column optionally includes one or more direct fusel oil side off-takes.

In yet another embodiment, the distillation system is one in which an aqueous, semi-crude methanol is taken as liquid above the feed in a single or rectifying column. The semi-crude methanol is passed to a rectifying column, from which methanol product is taken overhead or at a high level. Preferably, water or aqueous methanol is taken as a bottoms stream.

Alternatively, undesirable by-products are removed from the separated methanol stream from the methanol synthesis reactor by adsorption. In such a system, other components such as fusel oil can be recovered by regenerating the adsorbent.

### V. USE OF THE METHANOL COMPOSITION IN THE MANUFACTURE OF OLEFINS

The methanol product composition of this invention can be used as feed for any conventional process. Examples of such uses include the manufacture of methyl tertiary butyl alcohol (MTBE) for use in reformulated gasolines and oxygenated fuels, the use of methanol as a fuel for fuel cells, use as feedstock to make olefins, and for use in making acetic acid and formaldehyde.

The methanol product stream of this invention is particularly suited for conversion to olefins, particularly ethylene and/or propylene. The methanol product stream can be fed directly to an olefin conversion process or it can be transported in large quantities over great distances and converted to olefins.

According to this invention, the methanol product can be produced in large scale quantities for conversion to olefins, which is of great advantage for further conversion of the olefins to polyolefins such as polyethylene and polypropylene. Advantageously, this invention allows for at least 100,000 metric tons of methanol product per year. Preferably, production is at least 500,000 metric tons per year, more preferably at least 1 million metric tons per year, and most preferably at least 2 million metric tons per year.

In one embodiment, the methanol stream of the invention is separated from a crude methanol stream, and transported to a location geographically distinct from that where the methanol composition was separated from the crude methanol stream. Preferably, the methanol composition of this invention is loaded into a vessel, and the vessel is transported over a body of water to a storage facility. The methanol can be easily transported at least 100, 500 or 1,000 miles or more. Once arriving at the storage facility, the methanol composition is delivered to a storage tank. From the storage tank, the methanol composition is ultimately sent to an olefin conversion unit for conversion to an olefin product. The methanol composition is preferably, loaded onto a ship, with the ship able to contain at least 20,000 tons, preferably at least 40,000 tons, and more preferably at least 80,000 tons.

An advantage of being able to transport the methanol composition is that the units which produce the methanol do not have to be located in close geographic proximity to the olefin conversion unit. This makes it possible to use remote gas reserves. These remote gas reserves would be used as feed for the methanol manufacturing facility. The methanol made at these remote sites can then be easily transported to a suitable location for conversion to olefins. Since olefins and polyolefins (i.e., plastics) demands are typically low at the remote gas sites, there will generally be a desire to transport methanol to high olefins and plastic demand areas. Methanol is routinely transported in vessels that are similar to those that transport crude oil and other fuels. Examples of locations of remote gas reserves include the coastline of west Africa, northwest Australia, in the Indian Ocean, and the Arabian Peninsula. Examples of locations of preferred sites to convert methanol to other products such as olefins include the U.S. Gulf coast and northwest Europe.

### VI. EXAMPLES

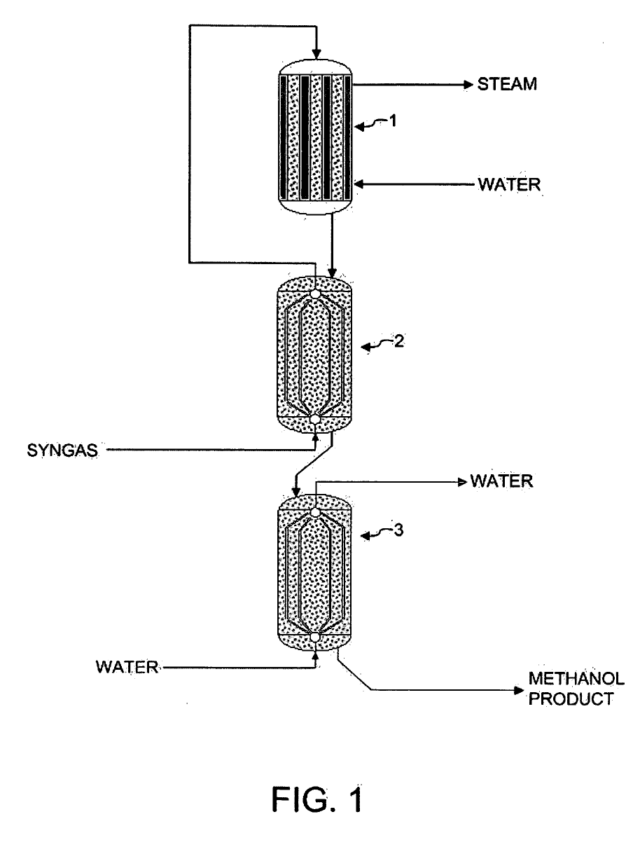

One example of the reaction system of this invention is shown in FIG. 1. In the embodiment of FIG. 1, syngas containing CO, CO₂ and hydrogen is sent to a second reactor 2, where the syngas is preheated and then sent to a first reactor 1. The heated syngas contacts catalyst in the reactor 1 to begin the conversion reaction, and hot effluent or product from the first reactor 1 is sent to the second reactor 2 to preheat the syngas. In the embodiment of FIG. 1, the syngas flows through tubes in the reactor 2 (tube side) and the effluent product from the reactor 1 contacts catalyst in a shell side of the reactor, continuing the conversion reaction.

Effluent product from reactor 2 is sent to a third reactor in series, reactor 3, as feed. Methanol product is then recovered from the reactor 3. The reactor 3 is cooled during the reaction process using liquid water as a cooling medium. The liquid water sent to the reactor 3 exits from the reactor 3 in the liquid phase.

### Example 1

The reaction system of FIG. 1 was simulated using a reactor kinetic and heat transfer model, described in Example 4. Syngas feed was set at 147° C. (entering reactor 2, prior to preheating), methanol product from reactor 3 was at 192° C., and system pressure was at 47 bar, with a 1 bar pressure drop across the system. Reactor 1 inlet temperature was 247° C., and reactor 1 outlet temperature was 263° C. Reactor 1 was cooled using medium pressure boiling water at 225° C. Reactor 2 was cooled using the effluent gas from Reactor 1. Reactor 3 was cooled using liquid water as a cooling medium. Water was input to the reactor 3 at a temperature of 128° C. and emerged from the reactor 3 at a temperature of 190° C. Additional reactor data is shown in Table 1.

### Example 2

A comparative example utilizing a single reactor with the same overall catalyst volume as that of the system in Example 1 was run. Water at the same temperature as reactor 1 of Example 1 (225° C.) was utilized as a cooling medium, with the water evaporating during the cooling process to form steam. All other feed conditions were identical to those of Example 1. The detailed reactor design data and results are shown in Table 1. The overall CO and CO₂ conversion in this example is 26.4%, much lower than that of Example 1. This can be attributed to the fact that the high level cooling medium at 225° C. results in a reactor effluent temperature that is higher than that of Example 1. The higher effluent temperature results in a reduced equilibrium conversion level.

### Example 3

A second comparative example utilizing a single reactor with the same overall catalyst volume as that of the system in Example 1 was run. This second comparative example was similar to that of Example 2, except that conversion is improved over that of Example 2 by using a lower temperature cooling medium. The reactor volume and other conditions were otherwise the same. The cooling in this example was also evaporative water cooling, with the water boiling at 168° C. Additional reactor design data and results are shown in Table 1.

The data in Table 1 show that the coolant temperature used in this example achieved the same reactor effluent temperature as that in Example 1 (192° C.). Thus, the equilibrium driving force at the reactor exit in this example is identical to that in Example 1. The overall conversion in this example is 33.2%, which is somewhat less than in Example 1. In addition, the fact that the heat of reaction is recovered by generating 168° C. steam instead of 225° C. steam means that the usefulness of the waste heat is substantially reduced.

TABLE 1

| | Example 1 | Example 2 | Example 3 |
|---|---|---|---|
| Reactor 1 | | | |
| Diameter | 5.5 | 5.69 | 5.69 |
| Height | 8 | 22 | 22 |
| Tube diameter | 1.575 | 1.575 | 1.575 |
| Tube spacing | 2.5 | 2.5 | 2.5 |
| Catalyst in/outside tubes | Inside | Inside | Inside |
| Coolant Temp in | 225 | 225 | 168 |
| Coolant Temp out | 225 | 225 | 168 |
| Reactor volume, m3 | 190.1 | 559.4 | 559.4 |
| Reactor 2 | | | |
| Diameter | 5.8 | | |
| Height | 7 | | |
| Tube diameter | 1.75 | | |
| Tube spacing | 2 | | |
| Catalyst in/outside tubes | Outside | | |
| Coolant Temp in | 147 | | |
| Coolant Temp out | 230 | | |
| Reactor volume, m3 | 184.9 | | |
| Reactor 3 | | | |
| Diameter | 5.8 | | |
| Height | 7 | | |
| Tube diameter | 1.5 | | |
| Tube spacing | 2.1 | | |
| Catalyst in/outside tubes | Outside | | |
| Coolant Temp in | 128 | | |
| Coolant Temp out | 190 | | |
| Reactor volume, m3 | 184.9 | | |
| Total All Reactors | | | |
| Reactor volume, m3 | 560.0 | 559.4 | 559.4 |
| Cox conversion | 35.3% | 26.4% | 33.2% |
| H2 conversion | 40.9% | 30.0% | 40.8% |

### Example 4

Temperature profiles, as well as zero and maximum rate temperature profiles, were calculated for each of Examples 1-3 using the kinetic model of Szarawa and Reichman, Int. Chem. Proc. 1 (1980) 331-343. Kinetic expressions were based on the following two independent reactions.
CO+2H₂ [symbol] CH₃OH (1)
CO₂+3H₂ [symbol] CH₃OH+H₂ (2)

The kinetic rate expressions were as follows:

$$\begin{matrix}{r}_{1} = 0.2032 exp ( \frac{- 2954}{T} ) {p}_{CO}^{0.5} {p}_{H 2} ( 1 - \frac{{p}_{CH 3 OH}}{{K}_{1} {p}_{CO} {p}_{H 2}^{2}} ) & ( 3 ) \\ {r}_{2} = 8.893 ⨯ {10}^{- 3} exp ( \frac{- 6163}{T} ) {p}_{CO 2}^{0.5} {p}_{H 2}^{1.5} ( 1 - \frac{{p}_{CH 3 OH} {p}_{H 2 O}}{{K}_{2} {p}_{CO 2} {p}_{H 2}^{3}} ) & ( 4 )\end{matrix}$$

Pressures are in bar and temperatures in ° K, and the reaction rate r is in mol.g⁻¹.hr⁻¹. The simulation model was programmed in MATLAB using the following 1-dimensional continuity and energy balance equations:

$$\begin{matrix}\frac{ⅆ {N}_{i}}{ⅆ z} = {θ}_{cat} {ρ}_{b} {\sum }_{j} {υ}_{ij} {r}_{j} {η}_{j} & ( 5 ) \\ {N}_{g} {C}_{p}^{o} \frac{ⅆ T}{ⅆ z} = {θ}_{cat} {ρ}_{b} {\sum }_{j} ( - Δ {H}_{j} ) {r}_{j} {η}_{j} + {A}_{surf}^{*} {U}_{o} ( {T}_{c} - T ) & ( 6 )\end{matrix}$$

where T_{c} is the local temperature of the coolant. The remainder of the nomenclature is as follows:

A*_{surf} surface:volume ratio of membrane or heat transfer surface

Cₚ^{o} molar heat capacity of gas mixture

ΔHⱼ heat of reaction for reaction j

kⱼ rate constant for reaction j

K_{eq,j} reaction equilibrium constant for reaction j,

$${K}_{eq , j} = exp ( \frac{- Δ {G}_{j}}{RT} )$$

Nᵢ molar flux of component i

N_{g} molar flux of total gas stream

pᵢ partial pressure of component i

rⱼ molar rate of reaction j based on mass of catalyst

R gas constant=8.314 J.mol⁻¹.° K⁻¹

T pseudo-homogeneous reaction temperature

Uₒ overall heat transfer coefficient

z axial dimension

The heats of reaction, equilibrium constants and heat capacities are functions of temperature and are calculated in the model using literature-based polynomial correlations. Such methods can be found, for example, in the book by R. C. Reid et al, The Properties of Gases & Liquids, 4th ed. 1987 New York: McGraw-Hill.

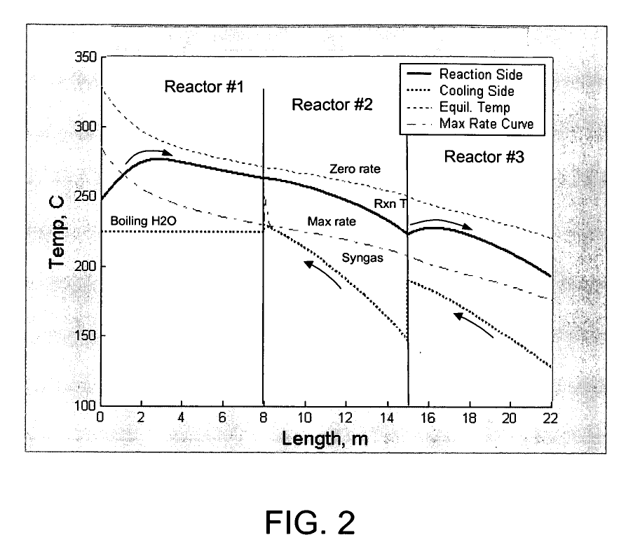

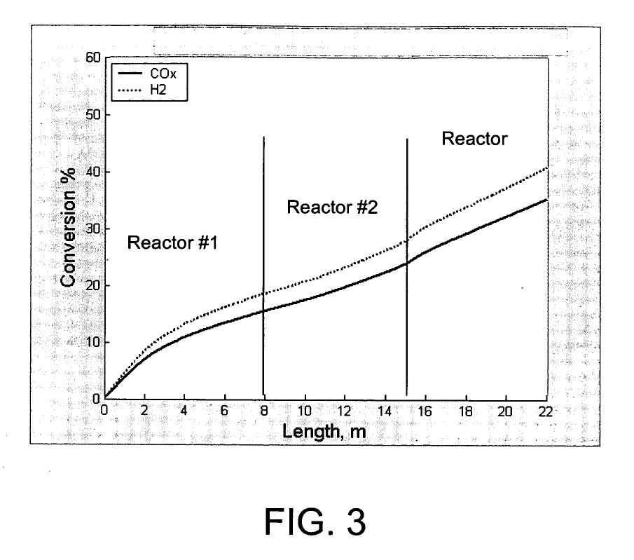

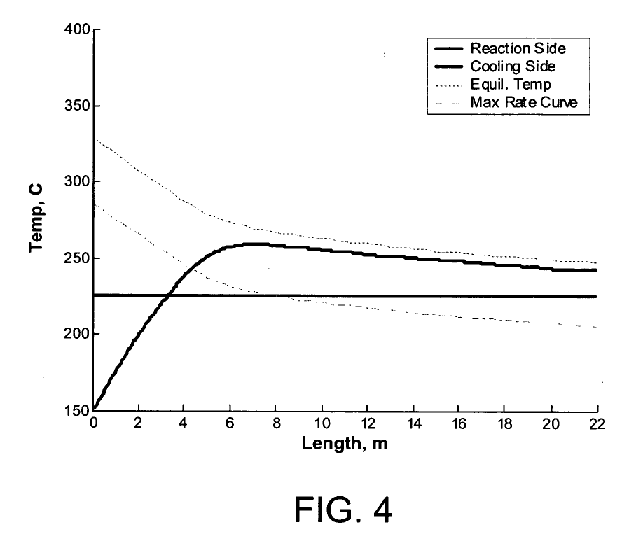

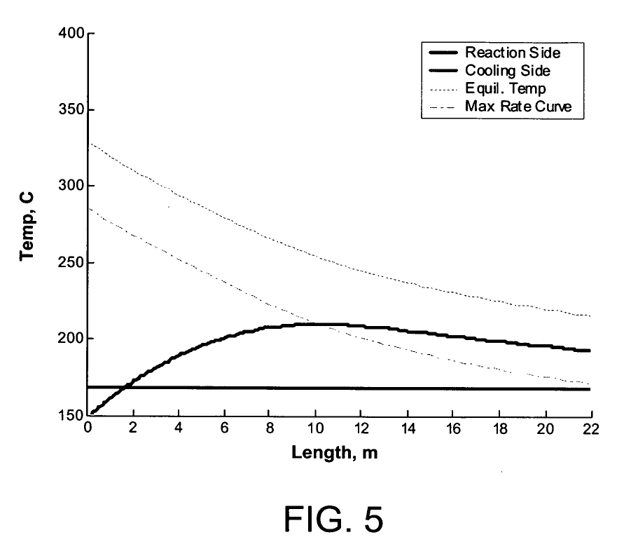

Temperature profiles for the three reactor system of Example 1 are shown in FIG. 2. FIG. 3 shows conversion profiles for the three reactor system of Example 1. FIG. 4 shows the temperature profile for the single reactor system of Example 2. FIG. 5 shows the temperature profile for the single reactor system of Example 3.

The principles and modes of operation of this invention have been described above with reference to various exemplary and preferred embodiments. As understood by those of skill in the art, the overall invention, as defined by the claims, encompasses other preferred embodiments not specifically enumerated herein.

What is claimed is:

1. A process for making methanol from syngas in a three reactor system, comprising:
feeding syngas to three reactors in series to form the methanol product; and,
cooling the reactors during the formation of the methanol product, wherein at least one of the reactors is cooled by evaporative cooling.

2. The process of claim 1, wherein at least one of the reactors is cooled by water evaporative cooling.

3. The process of claim 1, wherein at least one reactor is cooled by preheating the syngas feed.

4. The process of claim 1, wherein at least one reactor is cooled by water sensible cooling.

5. The process of claim 1, wherein one reactor is cooled by water evaporative cooling, one by preheating syngas feed, and one by water sensible cooling.

6. The process of claim 1, wherein the first of the three reactors in series is cooled by evaporative cooling.

7. The process of claim 6, wherein the first of the three reactors in series is cooled by water evaporative cooling.

8. The process of claim 1, wherein the third reactor in series is cooled by water sensible cooling.

9. The process of claim 1, wherein the second and third reactors in series are cooled by sensible cooling.

10. The process of claim 9, wherein the second reactor in series is cooled by gas sensible cooling.

11. The process of claim 10, wherein the second reactor in series is cooled by preheating the syngas feed to the first reactor.

12. The process of claim 1, wherein the first reactor has an inlet temperature of from 230° C. to 270° C.

13. The process of claim 1, wherein the first reactor has an outlet temperature of from 245° C. to 285° C.

14. The process of claim 1, wherein the outlet temperature of the second reactor is cooler than that of the first reactor.

15. The process of claim 14, wherein the outlet temperature of the second reactor is at least 10° C. cooler than that of the first reactor.

16. The process of claim 1, wherein the third reactor has an outlet temperature of from 170° C. to 210° C.

17. The process of claim 1, wherein the outlet temperature of the third reactor is cooler than that of the second reactor.

18. The process of claim 17, wherein the outlet temperature of the third reactor is at least 20° C. cooler than that of the second reactor.

19. The process of claim 1, wherein at least one reactor is a fixed bed reactor having multiple cooling tubes spaced within a catalyst bed.

20. The process of claim 1, wherein at least one reactor contains multiple tubes, each packed with catalyst and surrounded by a heat transfer medium.

21. The process of claim 1, wherein the process is carried out at a pressure of less than 110 bara.

22. The process of claim 1, wherein the process is carried out at a pressure of less than 90 bara.

23. The process of claim 1, wherein the process is carried out at a pressure of less than 70 bara.

24. A process for making methanol from syngas in a three reactor system, comprising contacting the syngas with methanol forming catalyst in three reactors in series, wherein the second and third reactors in series are cooled by sensible cooling and the first reactor is cooled by water evaporative cooling, with the designation of first, second and third reactors being based on the order in which catalytic conversion occurs.

25. The process of claim 24, wherein the second reactor in series is cooled by preheating the syngas feed to the first reactor.

26. A process for making methanol from syngas in a three reactor system, comprising:
contacting the syngas with methanol forming catalyst in a three reactor system, with the three reactors being in series; and
contacting each of the reactors with fluid to cool the reactors, wherein the second and third reactors in series are cooled by sensible cooling, the first reactor is cooled by water evaporative cooling, and the second reactor in series is cooled by preheating the syngas feed to the first reactor.
